(19) United States
(12) Patent Application Publication
(10) Pub. No.: US 2004/0035182 A1
(43) Pub. Date: Feb. 26, 2004

(54) Methods and systems for determining polarization of a gas based on electron paramagnetic resonance

(76) Inventor: Steve Kadlecek, Carrboro, NC (US)

Correspondence Address: Amersham Health, Inc., 101 Carnegie Center, Princeton, NJ, 08540, US

(21) Appl. No.: 10/438,465

(22) Filed: May 15, 2003

Related U.S. Application Data
(60) Provisional application No. 60/381,111, filed on May 16, 2002.

(51) Int. Cl. G01N 7/00; G01N 9/00; G01N 19/10; G01N 25/00; G01N 27/00; G01N 29/02; G01N 33/497

(52) U.S. Cl. 73/23.2

(57) ABSTRACT

Polarization of a target gas, such as a noble gas, may be determined by combining the gas with an alkali metal vapor. The strength of a magnetic field that is applied to the mixture may be varied and a plurality of resonant peaks of the alkali metal vapor may be determined. Polarization of the gas may be determined based on the plurality of resonant peaks of the alkali metal vapor. In other embodiments, the strength of the magnetic field may be held relatively constant and the resonant frequency of a tuned detection circuit that is responsive to the magnetic field may be varied.

### FIELD OF THE INVENTION

The present invention relates generally to the field of hyperpolarization of gases, such as noble gases, and, more particularly, to methods and systems for determining the polarization of a hyperpolarized gas.

### BACKGROUND OF THE INVENTION

It has recently been discovered that polarized inert noble gases can produce improved MRI images of certain areas and regions of the body, which have heretofore produced less than satisfactory images in this modality. Polarized helium-3 (“³He”) and xenon-129 (“¹²⁹Xe”) have been found to be particularly suited for this purpose. Unfortunately, the polarized state of the gases may be sensitive to handling and environmental conditions and may, undesirably, decay from the polarized state relatively quickly. Because of the sensitivity of a polarized gas and the potential influence on the strength of the obtained in vivo signal, it is generally desirable to monitor the polarization level of the gas at various times during the product's life. For example, in-process monitoring can indicate the polarization achieved during the optical pumping process or the polarization lost at certain phases of the life cycle process (so as to determine the remaining useable useable polarization of the polarized gas).

Hyperpolarizers are used to produce and accumulate polarized noble gases. Hyperpolarizes artificially enhance the polarization of certain noble gas nuclei (such as ¹²⁹Xe or ³He) over the natural or equilibrium levels, i.e., the Boltzmann polarization. Such an increase is generally desirable because it enhances and increases the MRI signal intensity, which may allow physicians to obtain better images of the substance in the body. See, e.g., U.S. Pat. No. 5,545,396 to Albert et al., the disclosure of which is hereby incorporated herein by reference as if set forth fully herein in its entirety.

To produce the hyperpolarized gas, the noble gas is typically blended with optically pumped alkali metal vapors such as rubidium (“Rb”). These optically pumped metal vapors collide with the nuclei of the noble gas and hyperpolarize the noble gas through a phenomenon known as “spin-exchange.” The “optical pumping” of the alkali metal vapor is produced by irradiating the alkali-metal vapor with circularly polarized light at the wavelength of the first principal resonance for the alkali metal (e.g., 795 nm for Rb). Generally stated, the ground state atoms become excited, and then subsequently decay back to the ground state. Under a modest magnetic field (10 Gauss), the cycling of atoms between the ground and excited states can yield nearly 100% polarization of the atoms in a few microseconds. This polarization is generally carried by the lone valence electron characteristics of the alkali metal. In the presence of non-zero nuclear spin noble gases, the alkali-metal vapor atoms can collide with the noble gas atoms in a manner in which the polarization of the valence electrons is transferred to the noble-gas nuclei through a mutual spin flip “spin-exchange.”

After the spin-exchange has been completed, the hyperpolarized gas is separated from the alkali metal prior to introduction into a patient (to form a non-toxic pharmaceutically acceptable product). Unfortunately, both during and after collection, the hyperpolarized gas can deteriorate or decay relatively quickly (lose its hyperpolarized state) and, therefore, is preferably handled, collected, transported, and stored with care. Proper handling of a hyperpolarized gas is generally important because of the sensitivity of the hyperpolarized state to environmental and handling factors and the potential for undesirable decay of the gas from its hyperpolarized state.

Conventionally, the level of polarization has been monitored at the polarization transfer process point (i.e., at the polarizer or optical cell) in a hyperpolarizer device or measured at a site remote from the hyperpolarizer after the polarized gas is dispensed from the hyperpolarizer. For example, for the latter, the polarized gas is directed to an exit or dispensing port on the hyperpolarizer and into two separate sealable containers, a gas delivery container, such as a bag, and a small (about 5 cubic centimeter) sealable glass bulb specimen container. This glass bulb specimen container may then be sealed at the hyperpolarizer site and carried away from the hyperpolarizer to a remotely located high-field NMR spectroscopy unit (4.7T) to determine the level of polarization achieved during the polarization process. See, e.g., J. P. Mugler, B. Driehuys, J. R. Brookeman et al., MR Imaging and Spectroscopy Using Hyperpolarized 129Xe Gas; Preliminary Human Results, Mag. Reson. Med. 37, 809-815 (1997).

As noted above, conventional hyperpolarizers may monitor the polarization level achieved at the polarization transfer process point, i.e., at the optical cell or optical pumping chamber. To do so, a small “surface” NMR coil may be positioned adjacent to the optical pumping chamber to excite and detect the gas therein and, thus, monitor the level of polarization of the gas during the polarization-transfer process. The small surface NMR coil will typically sample a smaller volume of the proximate polarized gas and thus have a longer transverse relaxation time (T₂*) compared to larger NMR coil configurations. A relatively large tip angle pulse can be used to sample the local-spin polarization. The large angle pulse will generally destroy the local polarization, but because the sampled volume is small compared to the total size of the container, it will not substantially affect the overall polarization of the gas.

Typically, the surface NMR coil is operably associated with low-field NMR detection equipment, which is used to operate the NMR coil and to analyze the detected signals. Examples of low-field NMR detection equipment used to monitor polarization at the optical cell and to record and analyze the NMR signals associated therewith include low-field spectrometers using frequency synthesizers, lock-in amplifiers, audio power amplifiers, and the like, as well as computers.

It is now known that on-board hyperpolarizer monitoring equipment no longer requires high-field NMR equipment, but instead may use low-field detection techniques to perform polarization monitoring for the optical cell at lower field strengths (e.g., 1-100G) than conventional high-field NMR techniques. This lower field strength allows correspondingly lower detection equipment operating frequencies, such as 1-400 kHz.

For applications where the entire hyperpolarized gas sample can be located inside the NMR coil, an adiabatic fast passage (“AFP”) technique has been used to monitor the polarization of the gas in this type of situation. Unfortunately, in many production-oriented situations, this technique is not desirable. For example, to measure the polarization in a one-liter patient dose bag, a relatively large NMR coil and spatially large magnetic field is needed.

These patents are hereby incorporated by reference as if set forth fully herein in their entirety. More recently, Saam et al. has proposed a low-frequency NMR circuit expressly for the on-board detection of polarization levels for hyperpolarized ³He at the optical cell inside the temperature-regulated oven, which encloses the cell. See Saam et al., Low Frequency NMR Polarimeter for Hyperpolarized Gases, Jnl. of Magnetic Resonance 134, 67-71 (1998). Others have used low-field NMR apparatus for on-board polarization measurement. See, U.S. Pat. No. 6,269,648 to Hasson et al., U.S. Pat. No. 6,237,363 to Zollinger et al., U.S. Pat. No. 6,295,834 to Driehuys, and U.S. Pat. Nos. 5,642,625 and 5,809,801 to Cates et al. the disclosures of which are hereby incorporated herein by reference as if set forth fully herein in their entireties.

The low-frequency NMR detection systems described above notwithstanding, there remains a need for improved methods and/or systems for efficiently and reliably determining and/or monitoring the level of polarization of polarized gases in various points in the production cycle.

### SUMMARY OF THE INVENTION

Methods and systems, in accordance with various embodiments of the present invention, may determine polarization of a target gas that is, for example, polarized by spin-exchange with an optically pumped alkali-metal vapor. In particular the resonant frequency, frequency width, and transition strength of transitions between the various hyperfine states of the alkali metal atom may be probed using radio frequency (RF) fields. These factors are indicative of the alkali metal polarization and/or the speed/rate at which the alkali metal vapor is being polarized. The eventual target gas polarization is proportional to the alkali metal polarization. The target gas polarization, however, typically builds up over the course of several hours, while the alkali metal polarization typically reaches equilibrium in less than a second. Thus, an alkali metal polarization determination may be useful in quickly evaluating the eventual polarization of the target gas and/or may allow early identification of abnormalities in the production environment, e.g., bad optical cell, laser misalignment, etc.)

According to certain embodiments of the present invention, polarization of a target gas may be determined by combining the gas with an alkali metal vapor. The strength of a magnetic field that is applied to the mixture may be varied and a plurality of resonant peaks of the alkali metal vapor may be determined. In other embodiments, the resonant frequency of a tuned detection circuit may be varied and the holding magnetic field may be held relatively constant. Polarization of the target gas may be evaluated based on the plurality of resonant peaks of the alkali metal vapor. In particular embodiments, the gas may comprise at least one of¹²⁹Xe and/or ³He, and the alkali metal vapor may comprise at least one of ⁸⁵Rb and/or ⁸⁷Rb. The strength of the magnetic field may vary between a range spanning at least 10% of the magnetic field strength.

In further embodiments of the present invention, the width of one or more of the resonant peaks may be determined and a time for the gas to reach a polarization threshold may be determined based on the width. The polarization threshold may be a projected final polarization level.

In still further embodiments of the present invention, the magnetic field that is applied to the gas and alkali metal vapor mixture may be increased and a first resonant peak of the alkali metal vapor determined. The spin of the gas atoms may be reversed, using, for example, an adiabatic fast passage process, and the magnetic field may be decreased. The magnetic field is increased again and a second resonant peak of the alkali metal vapor determined. A difference between frequencies associated with the first and second resonant peaks may be determined and the polarization of the gas may be determined based on this frequency difference.

### BRIEF DESCRIPTION OF THE DRAWINGS

Other features of the present invention will be more readily understood from the following detailed description of specific embodiments thereof when read in conjunction with the accompanying drawings, in which:

FIG. 1 is a block diagram that illustrates methods and systems for determining polarization of a gas in accordance with some embodiments of the present invention;

FIG. 2A depicts a container for holding a target gas.

FIG. 2B depicts an oven for receiving the container of FIG. 2A.

FIG. 2C is a schematic that illustrates an RF coil in a saddle configuration in accordance with some embodiments of the present invention;

FIG. 2D is an end view of the RF coil of FIG. 2C;

FIG. 3 is a graph that plots the homogeneity of the transverse magnetic field in the coil of FIG. 2C versus opening angle α in degrees;

FIG. 4 is a block diagram that illustrates data processing systems that may be used in the systems of FIG. 1 in accordance with some embodiments of the present invention;

FIG. 5 is a software architecture block diagram that illustrates methods and systems for determining polarization of a gas in accordance with some embodiments of the present invention;

FIG. 6 is a circuit diagram that illustrates the resonance effects of alkali metal atoms on the resonance of an oscillating circuit;

FIG. 7 shows the resonant frequency of the circuit of FIG. 6 as a function of the alkali metal atoms' resonant frequency;

FIG. 8 is a block diagram of an exemplary electron paramagnetic resonance (EPR) circuit that may be used in the systems of FIG. 1 in accordance with some embodiments of the present invention;

FIG. 9 is a graph that illustrates resonant peaks of an alkali metal vapor obtained using the EPR circuit of FIG. 8;

FIG. 10 is a block diagram of an EPR circuit that may be used in the systems of FIG. 1 in accordance with further embodiments of the present invention;

FIG. 11 is an exemplary schematic of the EPR circuit of FIG. 10 in accordance with some embodiments of the present invention;

FIGS. 12-14 are flowcharts that illustrate exemplary operations for determining polarization of a gas in accordance with some embodiments of the present invention; and

FIG. 15 is a flowchart that illustrates exemplary operations for producing a polarized gas in accordance with some embodiments of the present invention.

### DETAILED DESCRIPTION OF EMBODIMENTS OF THE INVENTION

While the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention as defined by the claims. Like reference numbers signify like elements throughout the description of the figures. In the figures, components, features, and/or layers may be exaggerated for clarity. Certain features or operations may be illustrated in broken line to indicate such feature or operation is optional.

As used herein, the terms “hyperpolarize,” “polarize,” and the like are used interchangeably and mean to artificially enhance the polarization of certain noble gas nuclei over the natural or equilibrium levels. Such an increase may be desirable because it may allow stronger imaging signals corresponding to better MRI images of a substance and/or a targeted area of a body. As is known by those of skill in the art, hyperpolarization can be induced by spin-exchange with an optically pumped alkali-metal vapor or alternatively by metastability exchange. See Albert et al., U.S. Pat. No. 5,545,396.

The present invention may be embodied as methods, systems, and/or computer program products. Accordingly, the present invention may be embodied in hardware and/or in software (including firmware, resident software, micro-code, etc.). Furthermore, the present invention may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

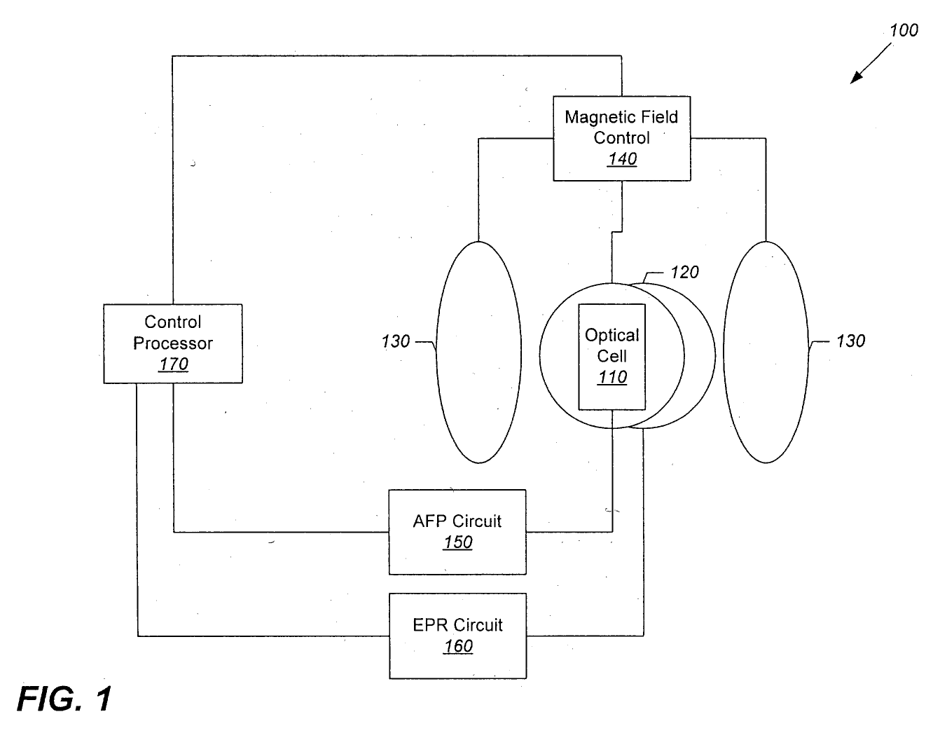

Referring now to FIG. 1, a system 100 for determining the polarization of a gas, in accordance with some embodiments of the present invention, will now be described. The system 100 comprises an optical cell 110 that contains a polarized target gas. In particular embodiments, the target gas may be a noble gas, such as ¹²⁹Xe or ³He. Other target gases and/or noble gases may also be used, alone or in combinations. Buffer gas formulations may also be used as described in the above-incorporated U.S. Pat. No. 6,295,834. The target gas may be polarized, for example, by an optically pumped spin-exchange with a vapor comprising an alkali metal, such as ⁸⁵Rb and/or ⁸⁷Rb. Other alkali metals may also be used, alone or in combinations. An exemplary list of alkali metals is provided in the above-incorporated U.S. Pat. No. 5,545,396 and U.S. Pat. No. 6,318,092, the disclosure of which is hereby incorporated herein by reference as if set forth fully herein in its entirety.

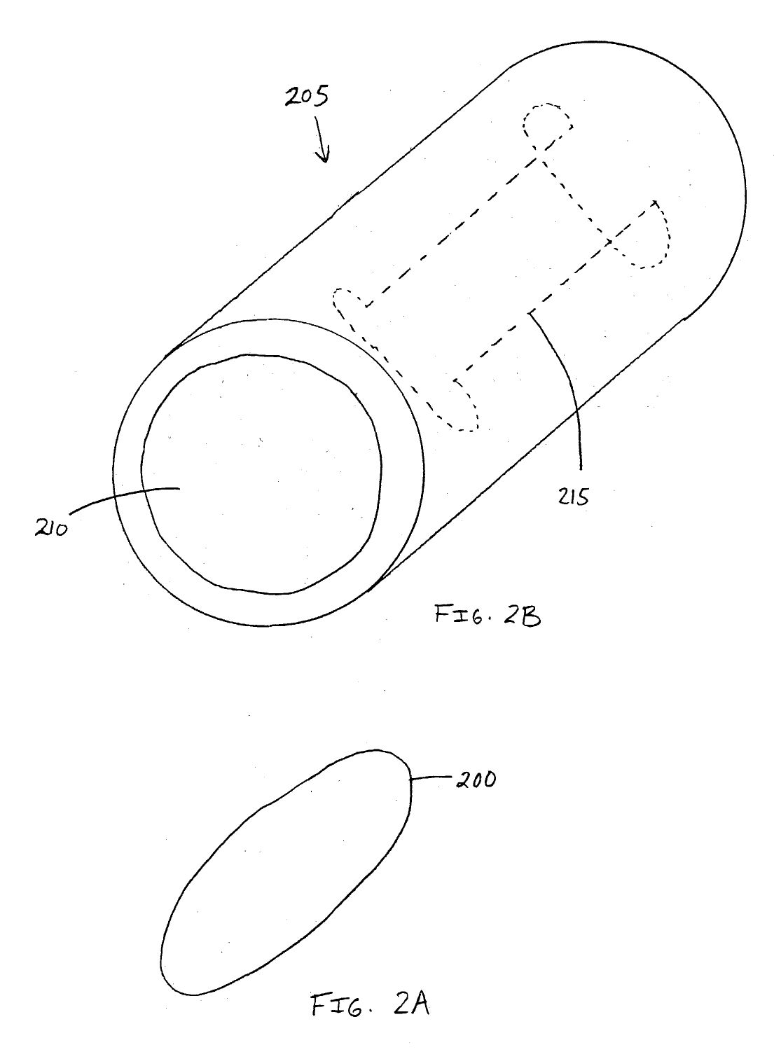

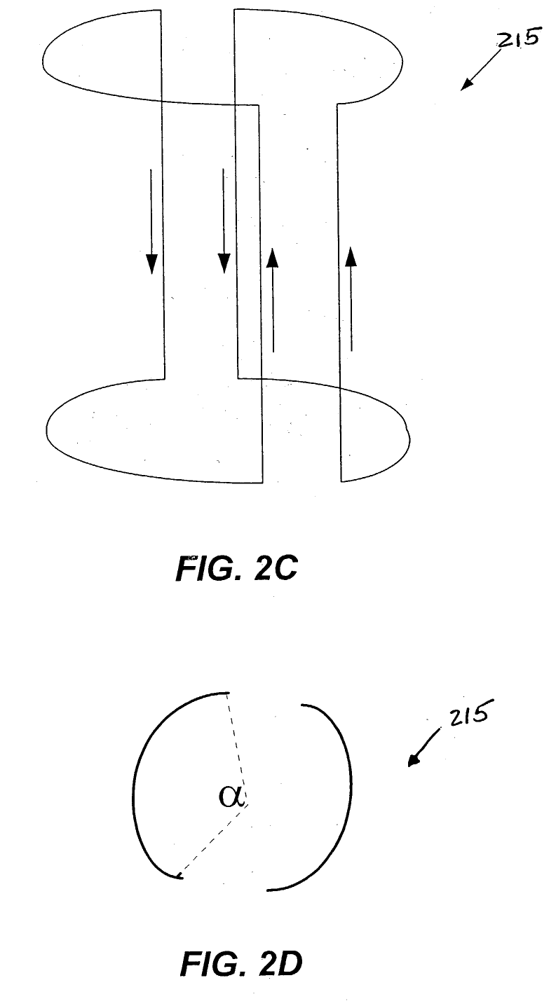

In accordance with particular embodiments illustrated in FIGS. 2A and 2B, the optical cell 110 may comprise a container 200, such as an aluminosilicate glass container, that may hold the target gas. The container may be configured to withstand up to 10 atmospheres of pressure and temperatures up to 200° C. This container 200 may be configured to be received in an oven 205, which is shown in FIG. 2B. The container 200 may be received through an end portion 210 of the oven. The end portion of the oven 205 may then be closed using a covering that is at least partially transparent to laser light. In other embodiments, the oven may have a window formed in a region of the oven body that is at least partially transparent to laser light. The coil 120 of FIG. 1 may be wound or formed on the body of the oven 205 as illustrated by coil 215 in FIG. 2B. The coil 215 may be configured to extend along substantially an entire length of the container 200 that is held within the oven.

In some embodiments, the oven 205 may comprise a non-metallic body having at least one window that is at least partially transparent to laser light. The oven 205 may have a substantially cylindrical extruded body and may, in some embodiments, comprise a ceramic material, such as cordierite. In particular embodiments, the oven 205 may have a diameter of about 5″, a length of about 6″, and a thickness of about 0.5″. The coil 215 can be wound directly on the oven and potted, for example, in MasterSil 801 sealant for improved strength and reduced susceptibility to shorting. In other embodiments, the coil 215 may be wound directly into machined or otherwise formed channels in the external surface of the body of the oven 205. The oven may be designed to be recirculating, small, stable, and conducive to optical pumping. The inside of the oven may be painted with infrared absorbing paint to reduce light scattering. The oven 205, container 200, and/or coil 215 assembly may be configured as a modular component for relatively easy replacement in a field setting.

The RF coil 215 may have a length of about 5.6″, a diameter of about 5″, and may comprise two turns of 18-gauge magnet wire. In particular embodiments shown in FIG. 2C, the RF coil 215 may comprise a saddle coil configuration. As shown in FIG. 2D, the saddle coil 215 has an opening angle α when viewed from an end portion thereof. Advantageously, the angle α may be selected to enhance the uniformity of the magnetic field in the optical pumping cell 110. The angle α may be determined by calculating the maximum magnetic field Bₘₐₓ and the minimum magnetic field Bₘᵢₙ in the optical pumping cell 110 and then varying the angle α to make the difference between the two relatively small. Bₘₐₓ and Bₘᵢₙ refer to the transverse component of the magnetic field in the optical pumping cell as that component is generally more involved in driving radio frequency transitions of the alkali metal atoms. In other embodiments, the RF coil 215 may be formed and/or wound on the body of the oven 205 in a “bird cage” configuration as is known to those of skill in the art.

In other embodiments, the oven 205 may be configured to hold the target gas without the use of the container 200 of FIG. 2A. In these embodiments, the oven 205 may be coated with an aluminosilicate sol gel to inhibit surface induced depolarization. Exemplary embodiments of sol-gel coated polarization vessels are described in U.S. patent application Ser. No. 09/485,476, the disclosure of which is hereby incorporated herein by reference as if set forth fully herein in its entirety.

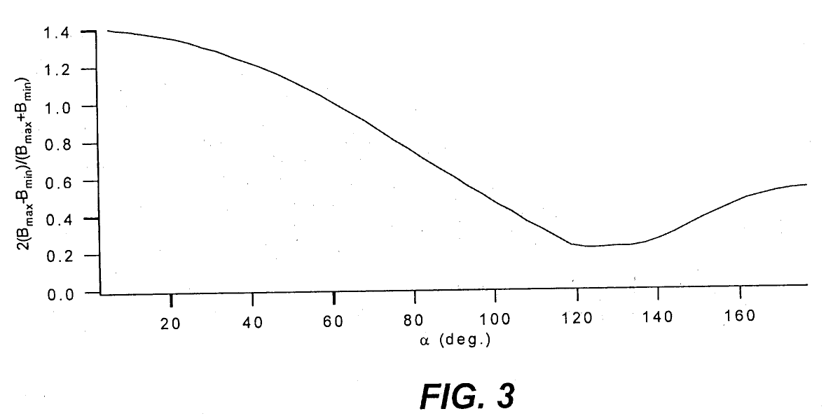

Referring now to FIG. 3, (Bₘₐₓ−Bₘᵢₙ)/((Bₘₐₓ+Bₘᵢₙ)/2) is plotted versus the angle α for an oven and coil 215 having the above-described specifications. For this coil geometry, the angle α may be adjusted to be about 120° to 130° to reduce variability in the magnetic field in the optical pumping cell 110.

Returning to FIG. 1, holding coils 130 may be used to applying a holding magnetic field to the gaseous mixture in the optical cell 110 at desired intervals during the optical pumping cycle. As will be discussed herein, achievable projected or actual polarization of the target gas may be determined based on various characteristics of resonant peaks of the alkali metal vapor in the optical pumping cell 110. One of these characteristics is the width of one or more of the resonant peaks of the alkali metal vapor.

Accordingly, in certain embodiments, it is generally desirable to reduce the magnetic field gradient in the optical cell 110, i.e., increase the homogeneity of the holding magnetic field. More specifically, if the holding field has a maximum value of Bₘₐₓ and a minimum value of Bₘᵢₙ and it is desired to measure the width δf a resonant peak at frequency f, then gradients are preferably limited such that Bₘₐₓ−Bₘᵢₙ<<Bₘᵢₙδf/f. In some embodiments, the holding field coils may be implemented as Helmholtz coils. An exemplary Helmholtz coil configuration is described in the above-incorporated U.S. Pat. No. 6,295,834 to Driehuys. To improve homogeneity of the holding field, however, an end compensated solenoid may be used to implement the holding coils 130 in accordance with other embodiments of the present invention. An exemplary end compensated solenoid is described in the above-incorporated U.S. Pat. No. 6,269,648 to Hasson et al.

The system 100 further comprises a magnetic field control module 140 that is coupled to the RF coil 120 and the magnetic holding field coils 130, an adiabatic fast passage (AFP) circuit 150 that is coupled to the optical cell 110, and an electron paramagnetic resonance (EPR) circuit 160 that is coupled to the coil 120. The AFP circuit 150 may be used to reverse the spin of the target gas atoms in the optical cell 110. AFP operations are described, for example, by M. V. Romalis and G. D. Cates in their 1998 paper entitled “Accurate 3He polarimetry using Rb Zeeman frequency shift due to the Rb-3He spin-exchange collisions,” Phys. Rev. A 58(4): 3004-3011, the disclosure of which is hereby incorporated herein by reference. The EPR circuit 160 may be used to facilitate the determination of a plurality resonant peaks of the alkali metal vapor in the optical cell 110 as will be described further hereinafter.

The magnetic field control module 140, AFP circuit 150, and the EPR circuit 160 may be coupled to a control processor 170 via, for example, a bus, a networked interface, a wireless interface, a wireline interface, and/or the like. The control processor 170 may be configured to determine the polarization of the target gas contained in the optical cell 110 based on a plurality of resonant peaks of the alkali metal vapor.

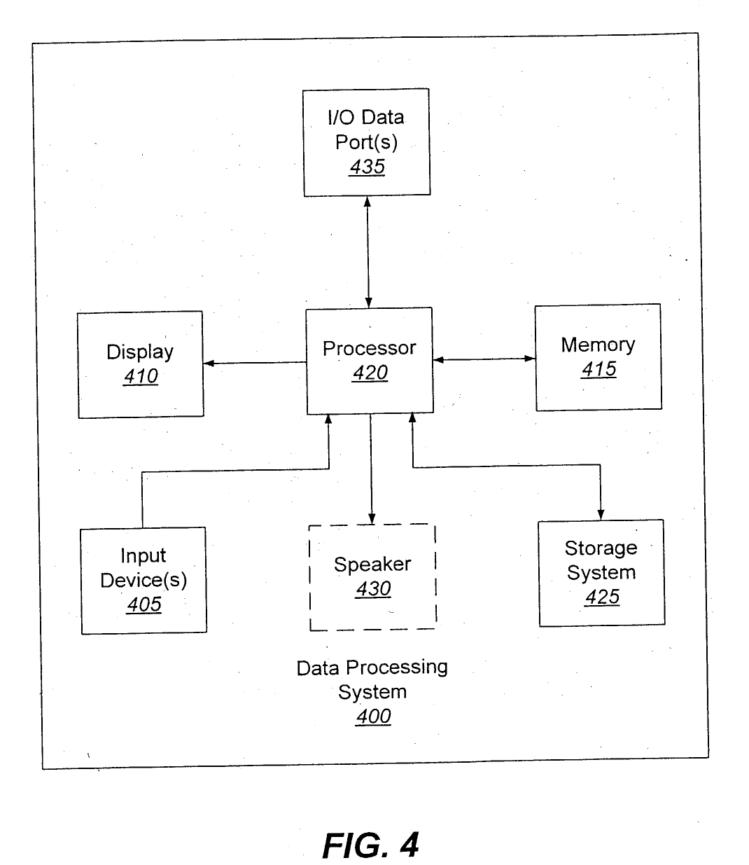

In accordance with some embodiments of the present invention, the control processor 170 may be embodied as a data processing system 400 as shown in FIG. 4 The data processing system 400 may include input device(s) 405, such as a keyboard or keypad, a display 410, and a memory 415 that communicate with a processor 420. The data processing system 400 may further include a storage system 425, a speaker 430, and an input/output (I/O) data port(s) 435 that also communicate with the processor 420. The storage system 425 may include removable and/or fixed media, such as floppy disks, ZIP drives, hard disks, or the like, as well as virtual storage, such as a RAMDISK. The I/O data port(s) 435 may be used to transfer information between the data processing system 400 and another computer system or a network (e.g., the Internet). These components may be conventional components such as those used in many conventional computing devices and/or systems, which may be configured to operate as described herein.

It will be further understood, however, that, in accordance with various embodiments of the present invention, the control processor 170 may be embodied as a stand alone computer or data processing system, an embedded processor system, an application specific integrated circuit, a programmed digital signal processor, a microcontroller, and/or the like.

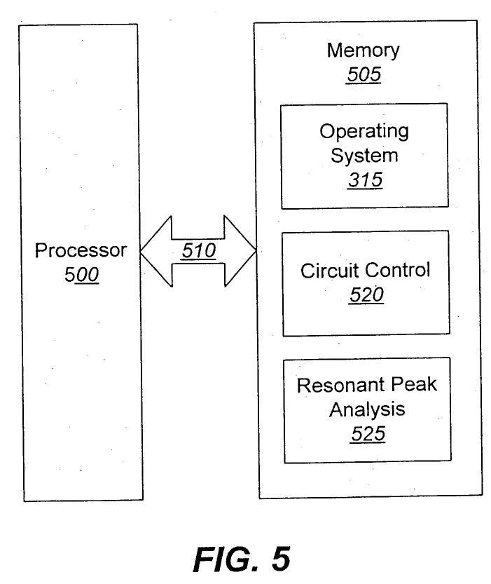

FIG. 5 illustrates a processor 500 and a memory 505 that may be used in some embodiments to provide the control processor 170 of FIG. 1, in accordance with the present invention. The processor 500 communicates with the memory 505 via an address/data bus 510. The processor 500 may be, for example, a commercially available or custom microprocessor. The memory 505 is representative of the memory devices containing the software and data used to determine the polarization of a gas in accordance with some embodiments of the present invention. The memory 505 may include, but is not limited to, the following types of devices: cache, ROM, PROM, EPROM, EEPROM, flash, SRAM, and DRAM.

As shown in FIG. 5, the memory 505 may contain up to three or more categories of software and/or data: the operating system 515, the circuit control module 520, and the signal strength analysis module 525. The operating system 515 controls the operation of the computer system. In particular, the operating system 515 may manage the computer system's resources and may coordinate execution of programs by the processor 500. The circuit control module 520 may be configured to generate the control operations of the magnetic field control module 140, the AFP circuit 150, and/or the EPR circuit 160 of FIG. 1. The resonant peak analysis module 525 may be configured to analyze electromagnetic signals that represent a plurality of resonant peaks of the alkali metal vapor contained in the optical cell 110 of FIG. 1 to determine the polarization of the target gas contained in the optical cell 110, in accordance with some embodiments of the present invention.

Although FIG. 5 illustrates an exemplary software architecture that may be used to determine polarization of a gas, in accordance with some embodiments of the present invention, it will be understood that the present invention is not limited to such a configuration but is intended to encompass any configuration capable of carrying out the operations described herein.

Computer program code for carrying out operations of the present invention may be written in an object-oriented programming language, such as Java, Smalltalk, or C++. Computer program code for carrying out operations of the present invention may also, however, be written in conventional procedural programming languages, such as the C programming language or compiled Basic (CBASIC). Other languages, such as LABVIEW, may also be used. Furthermore, some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

In accordance with some embodiments of the present invention, the system 100 of FIG. 1 may be used to determine or measure a plurality of a series of six resonance peaks of the alkali metal, which correspond to transitions between the neighboring hyperfine states of, for example, the ⁸⁵Rb atom electronic ground state. This choice may give a suitable signal-to-noise ratio, but the same thing could be done with other pairs of hyperfine states, or with the states of the ⁸⁷Rb atom, which is also present in the Rb vapor. As with NMR, the transition frequencies are largely determined by an externally applied magnetic field. Each resonance has a “strength,” which corresponds to the area under the resonance peak, a “width,” which is the frequency full-width-half-maximum of the resonance peak, and a “central frequency,” which is the frequency of maximum peak height for a given externally applied magnetic field.

In the presence of an applied magnetic field, and in the absence of other external perturbations, these resonances occur at six discrete, well-known frequencies. In more detail, the transitions between successive members of the ⁸⁵Rb electronic ground state F=3 and F=2 hyperfine manifolds are examined. The transition energy and splitting between successive ΔF=0, Δm=+/−1 transitions depends on the longitudinal field B_{z} as set forth below:

$$Δ {E}_{F , m - 1 / 2 \to F , m + 1 / 2} = {( - 1 )}^{F - 1} ( \frac{{B}_{z}}{G} ) ( 0.467 MHz ) [ 1 - 3.07 \times {10}^{- 4} m ( \frac{{B}_{z}}{G} ) ] \qquad EQ . 1$$

That is, the center of each manifold is at 0.467 MHz/G, and the splitting between subsequent transitions within a manifold is 143 Hz/G². The F=3 manifold has six transitions, and the F=2 manifold has four, but these are at the same frequency as the central four lines of the F=3 manifold, and, therefore, may be difficult to separate.

The strength of each resonance is proportional to the population difference between the two hyperfine states being driven. In more detail, the rate of RF power absorption when driving the transition between state |F,m> and state |F,m−1> depends on the population difference between the two states (ρ_{m,m}−ρ_{m-1, m-1}), the RF angular frequency detuning from line center Δ, the (collisionally or inhomogeneity broadened) linewidth γ, the RF field peak amplitude Bₓ, the number of alkali metal atoms N_{A}, and the RF angular frequency ω as follows:

$${N}_{A} ℏ ω \frac{{γ ( {g}_{s} {μ}_{B} {B}_{x} )}^{2}}{{Δ}^{2} + {γ}^{2} / 4} \frac{F ( F + 1 ) - m ( m - 1 )}{4 {( 2 I + 1 )}^{2}} ( {ρ}_{m , m} - {ρ}_{m - 1 , m - 1} ) \qquad EQ . 2$$

The spectrometer signal is proportional to this quantity if absorbed power is measured instead of frequency shift.

The RF signal may be swept across the first two peaks in the ⁸⁵Rb F=3 hyperfine manifold, and the area under each peak is proportional to the below equation:

(F(F+1)−m(m−1)(ρ_{m,m}−ρ_{m-1,m-1}) EQ. 3

In a spin-temperature distribution, the population difference between successive hyperfine levels is proportional to e^{βm}−e^{β(m-1)}=e^{β(m-1/2)}sin h(β/2). The first peak (⁸⁵Rb F=3,m=3⇄2) has area proportional to 6e^{5/2β} sin h(β/2). The second peak (the unresolved combination of ⁸⁵Rb F=3,m=2⇄1 and ⁸⁵Rb F=2,m=2⇄1) has area proportional to 10e^{3/2β} sin h(β/2)+14e^{3/2β} sin h(β/2)=14e^{3/2β} sin h(β/2), and their ratio R is 3/7 e^{β}. The polarization P=tan h(β/2). Therefore, polarization of the alkali metal vapor may be determined based on the ratio of the areas under two resonant peaks as set forth below:

$$P = \frac{7 R - 3}{7 R + 3} \qquad EQ . 4$$

The target gas spin polarization at saturation is proportional to the average alkali metal polarization. Therefore, by determining the average alkali metal polarization as described above, polarization of the target gas in the optical cell 110 (FIG. 1) may be determined. In particular, polarization of a target gas, such as He, may be approximated as set forth by the equation below:

$${P}_{He , sat} \approx 0.7 {P}_{Rb} \frac{[ Rb ] ⟨ {σ}_{SE} {v}_{He , Rb} ⟩}{[ Rb ] ⟨ {σ}_{SE} {v}_{He , Rb} ⟩ + 1 / {τ}_{w}} , \qquad EQ . 5$$

where P_{He,sat} is the saturation He polarization, P_{Rb} is the Rb polarization, [Rb] is the Rb density (the number of atoms per unit volume), σ_{SE} is the spin-exchange cross section, which has been measured to be about 6.7×10⁻²⁰ cm², ν_{HeRb} is the relative velocity between a typical He atom and a typical Rb atom as they collide, and τ_{W} is the relaxation time for He in the optical pumping cell.

The resonance peaks may be broadened by an alkali metal atom's interaction with its environment, such as, for example, collisions with other alkali metal atoms. The width of one or more resonant peaks may, therefore, be determined to obtain the collision frequency, which depends on the density of alkali metal atoms in the vapor phase. The rate at which the target gas approaches its saturation polarization depends on the alkali metal vapor density in a way that is understood, thereby allowing this diagnostic to predict how long it will take for the target gas to reach a predetermined polarization threshold.

The resonance peaks are shifted by collisions with the polarized target gas. This is described for example in the above-incorporated paper by Romalis and Cates. The change in central frequencies of the resonant peaks as the target gas polarization is suddenly changed may, therefore, be used to determine the target gas polarization. One approach for reversing the spins of the target gas atoms involves a technique known as “Adiabatic Fast Passage” (AFP). This may provide a relatively large frequency shift for a given target gas polarization, and, therefore, a suitable signal to noise ratio. The relationship between frequency shift and polarization may be represented as follows:

$$Δυ = \frac{16 π}{9} \frac{{μ}_{B}}{h} {κ}_{0} {μ}_{κ} [ He ] P , \qquad EQ . 6$$

where the frequency shift is ΔΛ, μ_{B}/h is the Bohr Magneton=1.4 MHz/G, μ_{K} is the nuclear magnetic moment of the gas being measured, [He] is the gas density (in this case, ³He density in atoms per unit volume), P is the gas polarization, and κ₀ is an enhancement factor that has been experimentally measured to be about 4.52+0.00934 T, where T is the temperature in celsius. The above-equation may also be used for Xe by experimentally determining the enhancement factor κ₀ for that gas.

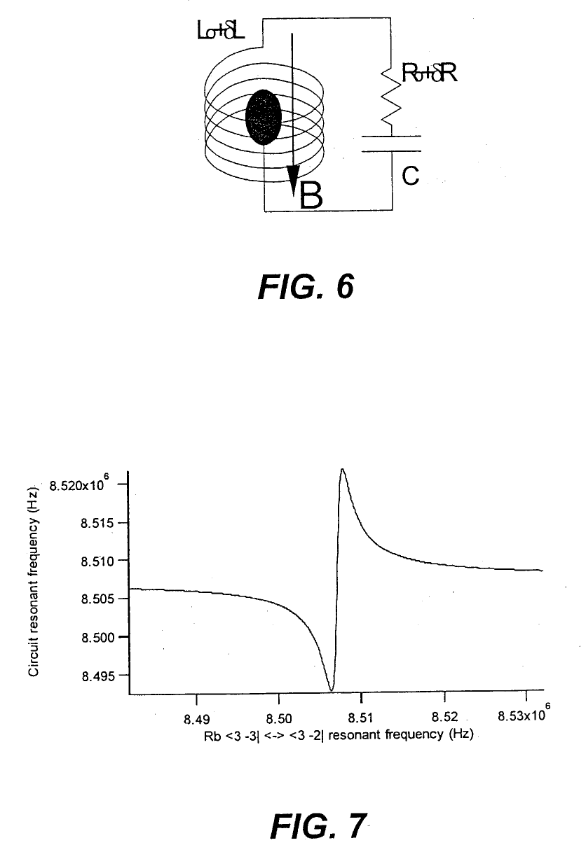

General principles underlying the EPR circuit 160 of FIG. 1 for determining the plurality of resonant peaks of the alkali metal vapor will now be described with reference to FIG. 6. A susceptibility measurement may be made by enclosing the optical cell 110 in a sensing coil 120, attaching the coil 120 to other elements to make a resonant circuit, and monitoring the characteristics of that circuit as the resonant frequency of either the circuit or the atoms is changed. The frequency of the atoms may be changed by varying the holding magnetic field that is applied thereto through the holding coils 130. The real part of the magnetic susceptibility and/or the imaginary part of the magnetic susceptibility may be measured. Depending on the resonant circuit chosen, these may be manifested in different ways. One approach, however, is illustrated in FIG. 6.

The alkali metal atoms (sphere) are enclosed in a coil that has inductance L₀ as long as the circuit resonance is far from the atoms' resonant frequencies. The circuit also has a small resistance, represented by R₀, which is mostly the resistance of the wire used to make the inductor. At high frequencies, this is typically much larger than the DC resistance of the wire. A capacitor of value C is added to make the circuit resonate, and the output voltage is measured across the capacitor. The properties of interest for this resonant circuit are its “quality factor” Q where

$$Q = \frac{1}{{R}_{0}} \sqrt{\frac{{L}_{0}}{C}} , \qquad EQ . 7$$

and its resonant frequency f₀, which may be approximated as

$${f}_{0} = \frac{1}{\sqrt{{L}_{0} C}} . \qquad EQ . 8$$

The quality factor is a measure of the power used to keep the circuit oscillating.

As the holding field B is changed to bring the alkali metal atoms' resonant frequencies near that of the circuit, the inductance of the coil changes because of the resonant action of the atoms. In more detail, near the |F=3, m=−3←→|F=3, m=−2> transition, the (complex) magnetic susceptibility may be referred to in terms of the real numbers χ and χ′.

$$\begin{matrix}\begin{matrix}χ = {χ}^{′} - ⅈ {χ}^{″} \\ where\end{matrix} & \\ {χ}^{″} ( Δ ) \approx \frac{{N ( {g}_{S} {μ}_{B} )}^{2}}{8 ℏ} {\int }_{0}^{∞} cos ( Δ {t}^{′} ) {ⅇ}^{- Γ {t}^{′}} ⅆ {t}^{′} = \frac{{N ( {g}_{S} {μ}_{B} )}^{2} Γ}{8 ℏ ( {Γ}^{2} + {Δ}^{2} )} & Eq . 9 \\ \begin{matrix}and \\ {χ}^{′} ( Δ ) = \frac{1}{π} {\int }_{- ∞}^{∞} \frac{{χ}^{″} ( {Δ}^{′} )}{{Δ}^{′} - Δ} ⅆ {Δ}^{′} \approx \frac{{N ( {g}_{S} {μ}_{B} )}^{2} Δ}{8 ℏ ( {Γ}^{2} + {Δ}^{2} )}\end{matrix} & Eq . 10\end{matrix}$$

Near resonance, the inductor is modified as follows:

L=L₀(1+μ₀χF) EQ. 11

where L₀ is the inductance far from resonance, and F is the “filling factor” of the optical cell 110 in the coil 120, which is approximately equal to the volume of the cell divided by the volume of the coil.

The effect of the atoms is therefore to increase L by δL=μ₀FL_{0χ′} and to increase R by δR=ωμ₀FL_{0χ″}. Therefore, the resonant frequency changes by approximately δf=f₀(δL)/(2L₀). Using the following exemplary values, FIG. 7 shows the resonant frequency of the circuit of FIG. 6 as a function of the alkali metal atoms'<3−3|→←<3−2| resonant frequency.

L₀=5 μH

C=70 pF

R=2□

N=2×10¹⁴ cm⁻³

F=1/6

Γ=5 kHz

g_{S}=2

μ_{B}=9.274×10⁻²⁴ J/T

μ₀=4π×10⁻⁷ N/A²

□=1.054×10⁻³⁴ JS

Because the inductance change is a complex value, it may be understood in the context of a change in the value of the inductor (the real part, δL) and an increase in the resistance (the imaginary part, which can be represented by δR). We can therefore detect this change by being sensitive to either the change in resonant frequency or the change in quality factor, in accordance with various embodiments of the present invention.

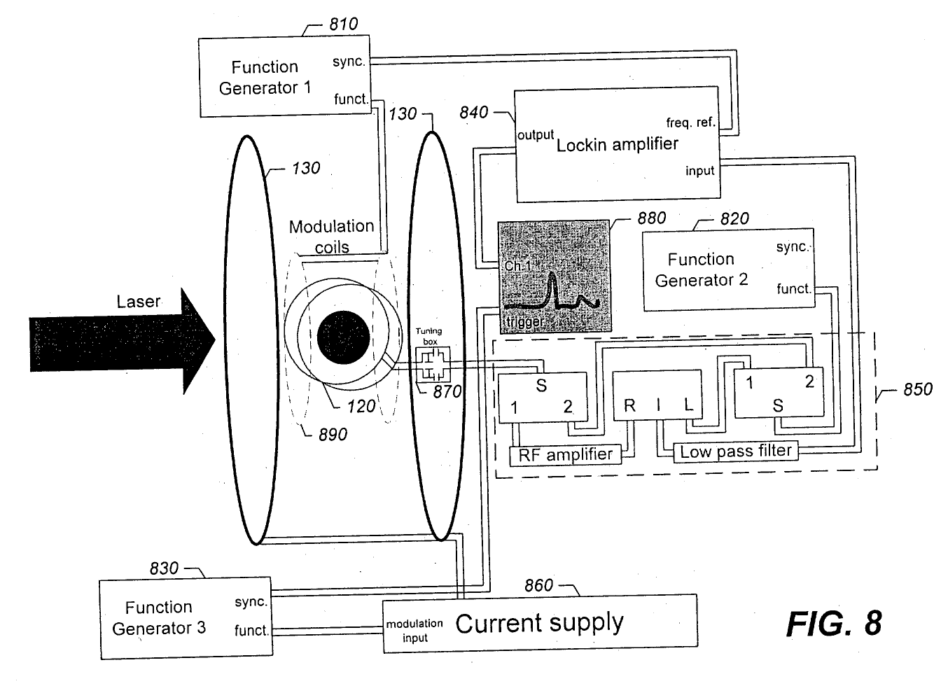

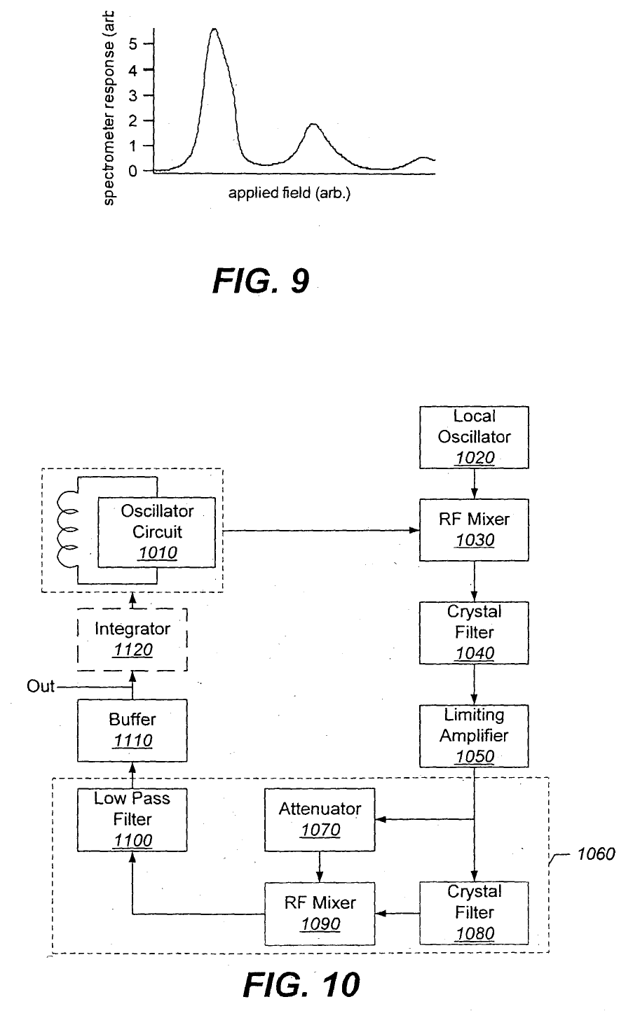

FIG. 8 illustrates particular embodiments of the EPR circuit 160 of FIG. 1 in which changes in a resonant circuit's quality factor may be detected to determine a plurality of resonant peaks of an alkali metal vapor. As shown in FIG. 8, the EPR circuitry comprises three function generators 810, 820, and 830, a lockin amplifier 840, RF power splitter circuitry 850, and a current supply 860. A tuning box 870, which may comprise one or more tuning capacitors, couples the RF power splitter circuitry 850 to the coil 120. The tuning box 870 may be configured to substantially match the impedance of the EPR circuitry to that of the coil 120 at resonance. The capacitance value to substantially match the impedance of the EPR circuitry with that of the coil 120 depends on the Q-value of the coil 120. Thus, changes in the effective Q-value of the coil 120 may be detected as a signal by the RF power splitter circuitry 850. The signal is amplified and mixed with an RF driving signal that is output from the function generator 820, filtered to remove the second harmonic frequency, and stored in an oscilloscope 880 for processing by the control processor 170 of FIG. 1. As the magnetic holding field is swept by the function generator 830 and the current supply 860, the effective Q-value of the coil 120 changes allowing the oscilloscope 880 to capture a signal that is representative of resonant peaks of the alkali metal vapor as shown in FIG. 9. Specifically, FIG. 9 shows the first two and part of a third of six ⁸⁵Rb peaks. The function generator 810, the modulation coils 890, and the lockin amplifier 840 may optionally be used to reduce low frequency noise.

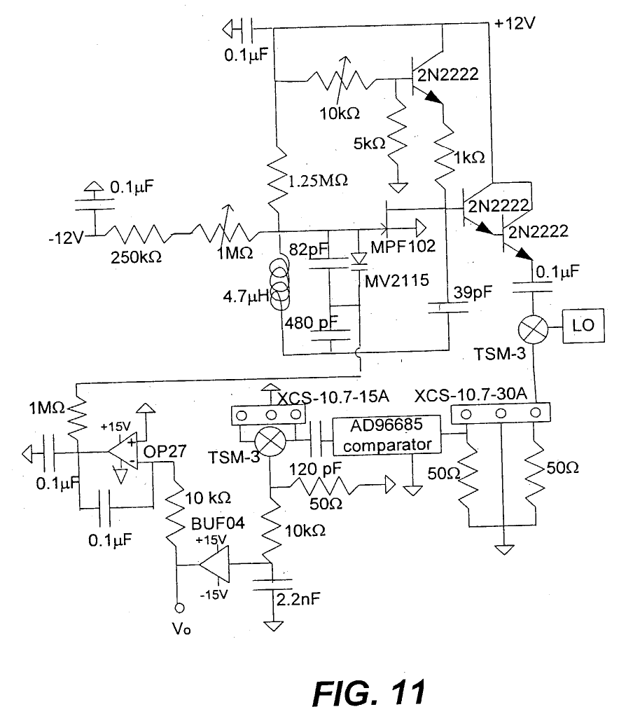

FIG. 10 illustrates other embodiments of the EPR circuit 160 of FIG. 1 in which resonant frequency changes may be determined directly. As shown in FIG. 10, a frequency modulation (FM) receiver circuit may be used to detect changes in frequency of a carrier signal, which is representative of the resonant peaks of the alkali metal vapor. In particular embodiments, the coil 120 of FIG. 1 is configured with an oscillator circuit 1010 to generate a modulating signal that may be used to modulate a carrier signal output from a local oscillator 1020 using an RF mixer 1030. In accordance with some embodiments of the present invention, the oscillator circuit may be a Colpitts oscillator. The output from the RF mixer 1030 is filtered by a crystal filter 1040 and then processed by a limiting amplifier 1050 where the signal is overdriven and subsequently clipped. The output of the limiting amplifier 1050 is processed by a discriminator 1060, which comprises an attenuator 1070, a crystal filter 1080, an RF mixer 1090, and a low pass filter 1100. The discriminator 1060 may be a Foster-Seeley discriminator, which converts frequency variations in the received FM signal to amplitude variations. These amplitude variations are rectified and filtered to provide a DC output voltage that varies in amplitude and polarity as the received FM signal varies in frequency. The output from the discriminator 1060 is received in a buffer 1110 where it may be provided to the control processor 170 of FIG. 1 for processing. Specifically, the output from the discriminator is representative of changes in frequency of the FM signal, which is representative of resonant peaks of the alkali metal vapor. Optionally, an integrator 1120 may be used to provide feedback to the oscillator circuit 1010 and coil 120 to reduce a DC offset that may be present at the output of the discriminator 1060. A detailed schematic of a particular embodiment of the FM receiver of FIG. 10 is shown in FIG. 11. The coil 120 is implemented as a 4.7 μH inductor in FIG. 11.

Although FIGS. 1, 8, 10, and 11 illustrate exemplary system architectures for determining polarization of a gas, it will be understood that the present invention is not limited to such configurations but is intended to encompass any configuration capable of carrying out the operations described herein.

The present invention is described hereinafter with reference to flowchart and/or block diagram illustrations of methods, systems, and computer program products in accordance with exemplary embodiments of the invention. It will be understood that each block of the flowchart and/or block diagram illustrations, and combinations of blocks in the flowchart and/or block diagram illustrations, may be implemented by computer program instructions and/or hardware operations. These computer program instructions may be provided to a processor of a general purpose computer, a special purpose computer, or other programmable data processing apparatus to produce a machine, such that the instructions, which execute via the processor of the computer or other programmable data processing apparatus, create means for implementing the functions specified in the flowchart and/or block diagram block or blocks.

These computer program instructions may also be stored in a computer usable or computer-readable memory that may direct a computer or other programmable data processing apparatus to function in a particular manner, such that the instructions stored in the computer usable or computer-readable memory produce an article of manufacture including instructions that implement the function specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the flowchart and/or block diagram block or blocks.

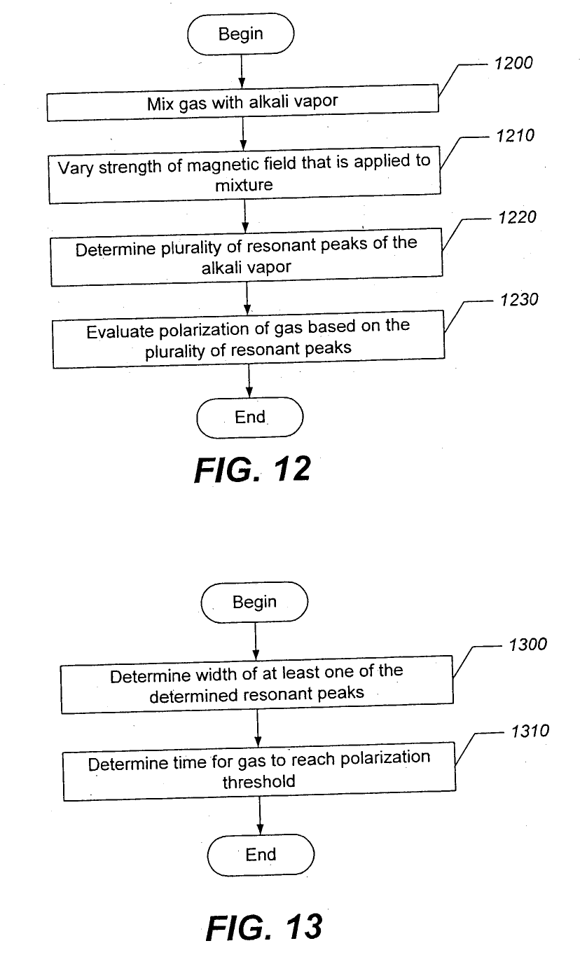

With reference to the flowcharts of FIGS. 12-14, exemplary operations of methods, systems, and computer program products for determining polarization of a gas, in accordance with some embodiments of the present invention, will be described hereafter.

Referring now to FIG. 12, operations begin at block 1200 where a target gas, such as ¹²⁹Xe and/or ³He, is mixed with an alkali metal vapor, such as ⁸⁵Rb and/or ⁸⁷Rb, in the optical cell 110 of FIG. 1. The magnetic field control module 140 of FIG. 1 is used to vary the strength of the holding field that is applied to the mixture at block 1210. The magnetic holding field may have a relatively low field strength that is less than about 100 gauss. In accordance with particular embodiments of the present invention, the magnetic field may be varied between values Bₘᵢₙ and Bₘₐₓ as set forth below:

Bₘᵢₙ=f(2.141−0.002f) EQ. 12

Bₘₐₓ=f(2.141+0.002f), EQ. 13

where Bₘᵢₙ and Bₘₐₓ are in gauss and f is the resonant frequency of the RF coil 120.

At block 1220, the EPR circuit 160 of FIG. 1 determines a plurality of resonant peaks of the alkali metal vapor. The EPR circuit 160 may be embodied as described above with reference to FIGS. 8, 10, and 11. The control processor 170 of FIG. 1 determines polarization of the target gas at block 1230 based on the plurality of resonant peaks, using, for example, the ratio of the areas under two resonant peaks as discussed above.

In particular embodiments of the present invention illustrated in FIG. 13, the control processor 170 of FIG. 1 at block 1300 may determine the width of one or more of the resonant peaks. Based on this width, the control processor 170 may determine a time for the target gas to reach a polarization threshold at block 1310, such as the ultimate achievable polarization level at the end of the polarization cycle.

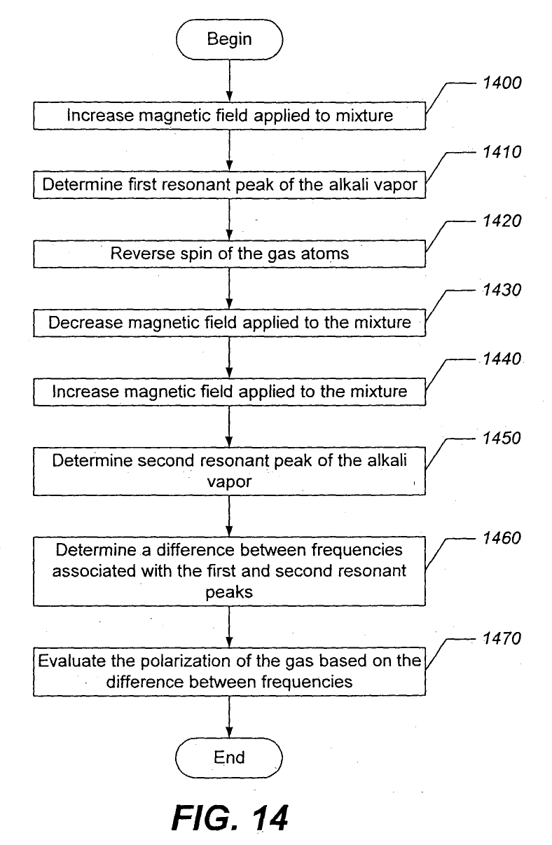

Referring now to FIG. 14, further embodiments of the present invention for determining polarization are illustrated. Operations begin at block 1400 where an on-board NMR reading may optionally be taken in the optical cell 110 and then the magnetic field control module 140 used to increase the magnetic field applied to the target gas and alkali metal vapor mixture in the optical cell 110. The magnetic field may be increased from a first strength to a second strength that span a range of about 10% to 20% of the holding field strength. In particular embodiments of the present invention, the magnetic field may be increased from about 18 gauss to about 24 gauss. The EPR circuit 160 of FIG. 1 may determine one or more resonant peaks of the alkali metal vapor at block 1410. Next, at block 1420, the AFP circuit 150 of FIG. 1 may be used to reverse the atomic spins of the target gas atoms when the magnetic field control module 140 decreases the magnetic field applied to the mixture at block 1430. For example, the magnetic field may be decreased from the second strength to the first strength discussed above. A second NMR reading may then be optionally taken in the optical cell 110. The magnetic field applied to the mixture may again be increased at block 1440 as described above with respect to block 1400. The EPR circuit 160 of FIG. 1 may determine one or more additional resonant peaks of the alkali metal vapor at block 1450. The control processor 170 of FIG. 1 may determine at block 1460 a difference between frequencies associated with one or more resonant peaks determined at block 1410 and the one or more resonant peaks determined at block 1450. At block 1470, the control processor 170 may determine the polarization of the target gas based on the difference between the frequencies. Advantageously, because the polarization determined at block 1470 is independent of the conventional on-board polarimetry circuitry used to determine polarization, the polarization determined at block 1470 may be used to improve calibration of the conventional on-board polarimetry circuitry, which may use, for example, an NMR surface coil.

Although described above with respect to embodiments in which polarization of a target gas is evaluated by varying the strength of a magnetic field that is applied to a mixture of the target gas and an alkali metal vapor and the determining the resonant peaks of the alkali metal vapor, it will be understood that, in accordance with other embodiments of the present invention, the resonant frequency of a tuned detection circuit, such as the circuits described hereinabove with respect to FIGS. 8 and 10, may also be varied and the holding magnetic field held relatively constant. For example, instead of varying the strength of the magnetic field as described with respect to block 1210 of FIG. 12 and/or blocks 1400, 1430, and 1440 of FIG. 14, the resonant frequency of the detection circuit.

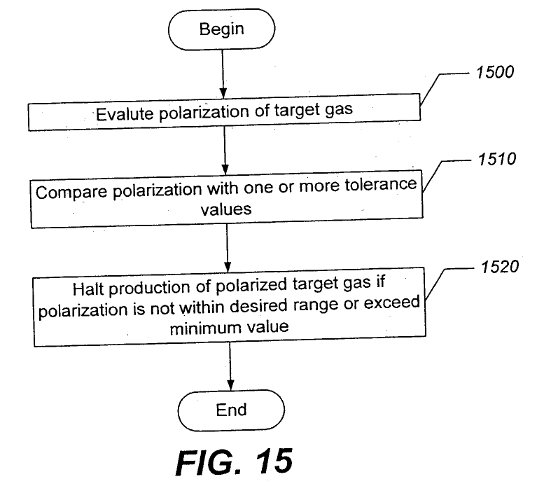

The system 100 may also be used in evaluating the integrity of a production system for a polarized gas. For example, by evaluating the polarization of a target gas based on resonant peaks associated with an alkali metal relatively early on during a production process, abnormalities, such as a bad container, oven, laser misalignment, etc, may be detected and corrective action taken. Referring now to FIG. 15, operations begin at block 1500 where polarization of a target gas is evaluated as discussed above, for example, with respect to FIGS. 12 and/or 14. At block 1510, the polarization of the target gas is compared with one or more tolerance values. If the projected polarization does not fall within a desired range or exceed a minimum value, for example, then production of the polarized gas may be halted at block 1520 and corrective action taken.

The flowcharts of FIGS. 12-15 illustrate the architecture, functionality, and operations of embodiments of the system 100 software. In this regard, each block represents a module, segment, or portion of code, which comprises one or more executable instructions for implementing the specified logical function(s). It should also be noted that in some alternative implementations, the function(s) noted in the blocks may occur out of the order noted in FIGS. 12-14. For example, two blocks shown in succession may, in fact, be executed substantially concurrently or the blocks may sometimes be executed in the reverse order, depending on the functionality involved.

Many variations and modifications can be made to the preferred embodiments without substantially departing from the principles of the present invention. All such variations and modifications are intended to be included herein within the scope of the present invention, as set forth in the following claims. In the claims, means-plus-function clauses are intended to cover the structures described herein as performing the recited function and not only structural equivalents, but also equivalent structures.

I claim:

1. A method of determining polarization of a target gas, comprising:
combining the target gas with an alkali metal vapor;
varying a strength of a magnetic field that is applied to the gas and alkali metal vapor mixture;
determining a plurality of resonant peaks of the alkali metal vapor using a radio frequency (RF) detection circuit; and
evaluating the polarization of the target gas based on the plurality of resonant peaks of the alkali metal vapor.

2. The method of claim 1, wherein the target gas comprises at least one of ¹²⁹Xe and ³He, and the alkali metal vapor comprises at least one of ⁸⁵Rb and ⁸⁷Rb.

3. The method of claim 1, wherein varying the strength of the magnetic field comprises:
varying the magnetic field between a range spanning at least 10% of the magnetic field strength.

4. The method of claim 1, further comprising:
determining a width of a respective one of the plurality of resonant peaks; and
determining a time for the target gas to reach a polarization threshold based on the width.

5. The method of claim 4, wherein the polarization threshold is a projected final polarization level.

6. The method of claim 1, wherein varying the strength of the magnetic field and determining the plurality of resonant peaks comprises:
increasing the magnetic field that is applied to the target gas and alkali metal vapor mixture;
determining a first resonant peak of the alkali metal vapor;
reversing a spin of the target gas atoms while decreasing the magnetic field that is applied to the target gas and alkali metal vapor mixture;
increasing the magnetic field that is applied to the target gas and alkali metal vapor mixture;
determining a second resonant peak of the alkali metal vapor;
determining a difference between frequencies associated with the first and second resonant peaks of the alkali metal vapor; and
determining the polarization of the target gas based on the difference between frequencies.

7. The method of claim 6, wherein reversing the spin of the target gas atoms comprises:
reversing the spin of the target gas atoms using an adiabatic fast passage process.

8. A method of determining polarization of a target gas, comprising:
combining the target gas with an alkali metal vapor, the target gas and the alkali metal vapor mixture being in electromagnetic communication with a resonant circuit having a Q-value associated therewith;
varying a strength of a magnetic field that is applied to the target gas and alkali metal vapor mixture;
detecting at least one change in the Q-value associated with the resonant circuit responsive to varying the strength of the magnetic field;
determining a plurality of resonant peaks of the alkali metal vapor based on the at least one change in the Q-value associated with the resonant circuit; and
evaluating the polarization of the target gas based on the plurality of resonant peaks of the alkali metal vapor.

9. The method of claim 8, wherein the resonant circuit comprises a RF detection coil and a capacitor and has a resistance associated therewith, and wherein detecting the at least one change in the Q-value comprises:
detecting at least one of a change in inductance of the RF detection coil and a change in the resistance of the resonant circuit.

10. The method of claim 8, wherein the target gas comprises at least one of ¹²⁹Xe and ³He, and the alkali metal vapor comprises at least one of ⁸⁵Rb and ⁸⁷Rb.

11. The method of claim 8, wherein varying the strength of the magnetic field comprises:
varying the magnetic field between a range spanning at least 10% of the magnetic field strength.

12. The method of claim 8, further comprising:
determining a width of a respective one of the plurality of resonant peaks; and
determining a time for the target gas to reach a polarization threshold based on the width.

13. The method of claim 12, wherein the polarization threshold is a projected final polarization level.

14. The method of claim 8, wherein varying the strength of the magnetic field, detecting the at least one change in the Q-value associated with the resonant circuit, and determining the plurality of resonant peaks comprises:
increasing the magnetic field that is applied to the target gas and alkali metal vapor mixture;
detecting a first change in the Q-value associated with the resonant circuit responsive to increasing the strength of the magnetic field;
determining a first resonant peak of the alkali metal vapor based on the first change in the Q-value associated with the resonant circuit;
reversing a spin of the target gas atoms while decreasing the magnetic field that is applied to the target gas and alkali metal vapor mixture;
increasing the magnetic field that is applied to the target gas and alkali metal vapor mixture;
detecting a second change in the Q-value associated with the resonant circuit responsive to increasing the strength of the magnetic field;
determining a second resonant peak of the alkali metal vapor based on the second change in the Q-value associated with the resonant circuit;
determining a difference between frequencies associated with the first and second resonant peaks of the alkali metal vapor; and
determining the polarization of the target gas based on the difference between frequencies.

15. The method of claim 14, wherein reversing the spin of the target gas atoms comprises:
reversing the spin of the target gas atoms using an adiabatic fast passage process.

16. A method of determining polarization of a target gas, comprising:
combining the target gas with an alkali metal vapor, the target gas and the alkali metal vapor mixture being in electromagnetic communication with a RF detection coil;
varying a strength of a magnetic field that is applied to the target gas and alkali metal vapor mixture;
inducing an electrical signal in the RF detection coil responsive to varying the strength of the magnetic field;
frequency modulating a carrier signal with the induced electrical signal;
detecting at least one change in frequency of the carrier signal;
determining a plurality of resonant peaks of the alkali metal vapor based on the at least one change in the frequency of the carrier signal; and
evaluating the polarization of the target gas based on the at the plurality of resonant peaks of the alkali metal vapor.

17. The method of claim 16, wherein the target gas comprises at least one of ¹²⁹Xe and ³He, and the alkali metal vapor comprises at least one of ⁸⁵Rb and ⁸⁷Rb.

18. The method of claim 16, wherein varying the strength of the magnetic field comprises:
varying the magnetic field between a range spanning at least 10% of the magnetic field strength.

19. The method of claim 16, further comprising:
determining a width of a respective one of the plurality of resonant peaks; and
determining a time for the target gas to reach a polarization threshold based on the width.

20 The method of claim 19, wherein the polarization threshold is a projected final polarization level.

21. The method of claim 16, wherein varying the strength of the magnetic field, detecting the at least one change in frequency, and determining the plurality of resonant peaks comprises:
increasing the magnetic field that is applied to the target gas and alkali metal vapor mixture;
detecting a first change in frequency of the carrier signal;
determining a first resonant peak of the alkali metal vapor based on the first change in the frequency of the carrier signal;
reversing a spin of the target gas atoms while decreasing the magnetic field that is applied to the target gas and alkali metal vapor mixture;
increasing the magnetic field that is applied to the target gas and alkali metal vapor mixture;
detecting a second change in frequency of the carrier signal;
determining a second resonant peak of the alkali metal vapor based on the second change in the frequency of the carrier signal;
determining a difference between frequencies associated with the first and second resonant peaks of the alkali metal vapor; and
determining the polarization of the target gas based on the difference between frequencies.

22. The method of claim 21, wherein reversing the spin of the target gas atoms comprises:
reversing the spin of the target gas atoms using an adiabatic fast passage process.

23. A system for determining polarization of a target gas, comprising:
an optical cell containing a mixture of the target gas with an alkali metal vapor;
an electromagnetic transmission device that is configured to vary a strength of a magnetic field that is applied to the mixture;
a circuit that is coupled to the optical cell and is configured to determine a plurality of resonant peaks of the alkali metal vapor; and
a control processor that is configured to determine the polarization of the target gas based on the plurality of resonant peaks of the alkali metal vapor.

24. The system of claim 23, wherein the mixture generates an electromagnetic signal responsive to the magnetic field that is applied thereto, and wherein the circuit comprises:
a RF coil that is wrapped around the optical cell and is configured to induce an electrical signal therein responsive to an electromagnetic signal generated by the mixture;
at least one tuning capacitor that is coupled to the RF coil and is configured to substantially match an impedance of the circuit with an impedance of the RF coil; and
the circuit being further configured to determine the plurality of resonant peaks of the alkali metal vapor based on the electrical signal induced in the RF coil.

25. The system of claim 23, wherein the optical cell comprises:
a non-metallic oven having a substantially cylindrical body; and wherein the circuit comprises:
an RF coil that is attached to the oven and extends about a major portion of the length of the oven body.

26. The system of claim 25, wherein the oven body is ceramic.

27. The system of claim 25, wherein the RF coil is a saddle coil that has an opening angle when viewed from an end portion thereof.

28. The system of claim 27, wherein the opening angle of the saddle coil is in a range of about 120° to 130°.

29. The system of claim 25, wherein the RF coil comprises two turns of 18 gauge magnet wire.

30. The system of claim 23, further comprising:
an end compensated solenoid that is configured to apply an electromagnetic holding field to the mixture.

31. The system of claim 23, wherein the mixture generates an electromagnetic signal responsive to the magnetic field that is applied thereto, and wherein the circuit comprises:
a RF coil that is disposed about the optical cell and is configured to induce an electrical signal therein responsive to an electromagnetic signal generated by the mixture;
an oscillator circuit that is coupled to the RF coil and is configured to generate a modulated carrier signal responsive to the electromagnetic signal generated by the mixture;
an RF receiver circuit that is configured to detect at least one change in frequency of the carrier signal; and
the circuit being further configured to determine a plurality of resonant peaks of the alkali metal vapor based on the at least one change in the frequency of the carrier signal.

32. An optical pumping cell for a polarized target gas, comprising:
a non-metallic container for the polarized target gas; and
a RF coil that is disposed about the container and has a saddle configuration that has an opening angle when viewed from an end portion thereof.

33. The optical pumping cell of claim 32, wherein the opening angle of the saddle coil is in a range of about 120° to 130°.

34. The optical pumping cell of claim 32, wherein the optical pumping cell comprises an oven that is configured to hold the non-metallic container therein.

35. The optical pumping cell of claim 33, wherein the oven comprises ceramic.

36. The optical pumping cell of claim 32, wherein the RF coil comprises two turns of 18 gauge magnet wire.

37. A system for determining polarization of a target gas, comprising:
means for combining the target gas with an alkali metal vapor;
means for varying a strength of a magnetic field that is applied to the target gas and alkali metal vapor mixture;
means for determining a plurality of resonant peaks of the alkali metal vapor; and
means for evaluating the polarization of the target gas based on the plurality of resonant peaks of the alkali metal vapor.

38. The system of claim 37, wherein the target gas comprises at least one of ¹²⁹Xe and ³He, and the alkali metal vapor comprises at least one of ⁸⁵Rb and ⁸⁷Rb.

39. The system of claim 37, wherein the means for varying the strength of the magnetic field comprises:
means for varying the magnetic field between between a range spanning at least 10% of the magnetic field strength.

40. The system of claim 37, further comprising:
means for determining a width of a respective one of the plurality of resonant peaks; and
means for determining a time for the target gas to reach a polarization threshold based on the width.

41. The system of claim 40, wherein the polarization threshold is a projected final polarization level.

42. A method of determining polarization of a target gas, comprising:
combining the target gas with an alkali metal vapor;
applying a magnetic field to the gas and alkali metal vapor mixture;
varying a resonant frequency of a radio frequency (RF) detection circuit that is responsive to the magnetic field;
determining a plurality of resonant peaks of the alkali metal vapor using the RF detection circuit; and
evaluating the polarization of the target gas based on the plurality of resonant peaks of the alkali metal vapor.

43. The method of claim 42, wherein the target gas comprises at least one of ¹²⁹Xe and ³He, and the alkali metal vapor comprises at least one of ⁸⁵Rb and ⁸⁷Rb.

44. The method of claim 42, further comprising:
determining a width of a respective one of the plurality of resonant peaks; and
determining a time for the target gas to reach a polarization threshold based on the width.

45. A method of producing a polarized gas, comprising:
combining a target gas with an alkali metal vapor;
determining a plurality of resonant peaks of the alkali metal vapor using the RF detection circuit;
evaluating the polarization of the target gas based on the plurality of resonant peaks of the alkali metal vapor;
comparing the polarization of the target gas with at least one polarization tolerance value; and
determining whether to proceed with production of the polarized target gas based on the comparison of the polarization of the target gas with the at least one polarization tolerance value.
